(19) United States
(12) Patent Application Publication
(10) Pub. No.: US 2024/0391992 A1
(43) Pub. Date: Nov. 28, 2024

(54) ANTIBODIES TARGETING XCL1 AND METHODS OF USING THE SAME

(71) Applicant: ELI LILLY AND COMPANY, Indianapolis, IN (US)

(72) Inventors: Stephanie Lynn BEASLEY, Greenwood, IN (US); Patrick Francis GREALISH, Indianapolis, IN (US); Jingxing LI, San Diego, CA (US); Chad D. PAAVOLA, San Diego, CA (US); Joshua Dade POORBAUGH, New Palestine, IN (US); Matthew Alan RENSCHLER, Indianapolis, IN (US); Ying TANG, San Diego, CA (US); Mark Andrew WORTINGER, Greenwood, IN (US)

(21) Appl. No.: 18/673,584

(22) Filed: May 24, 2024

Related U.S. Application Data
(60) Provisional application No. 63/504,051, filed on May 24, 2023.

(51) Int. Cl. C07K 16/24; A61K 39/00; A61P 37/06

(52) U.S. Cl. CPC C07K 16/24; A61P 37/06; A61K 2039/505; C07K 2317/33; C07K 2317/565

(57) ABSTRACT

The present invention provides compounds and methods targeting human lymphotactins, including therapeutic antibodies, pharmaceutical compositions and methods of use thereof, useful in the field of immune-mediated diseases including ulcerative colitis, vitiligo, multiple sclerosis, alopecia areata, psoriasis, type 1 diabetes, and asthma.

### SEQUENCE LISTING FILE

The present application is being filed along with a Sequence Listing in ST.26 XML format. The Sequence Listing is provided as a file titled “30574_SequenceListing” created May 6, 2024, and is 43 kilobytes in size. The Sequence Listing information in the ST.26 XML format is incorporated herein by reference in its entirety.

### FIELD OF THE INVENTION

The present invention is in the field of medicine. More particularly, the present invention relates to antibodies directed against lymphotactins, in particular the human chemokines XCL1 and XCL2; pharmaceutical compositions comprising such antibodies; and methods of using such antibodies. The antibodies disclosed herein bind and neutralize XCL1 and XCL2. The antibodies and methods of the present invention are expected to be useful in the field of autoimmune and chronic inflammatory diseases (collectively referred to herein as, immune-mediated diseases), particularly diseases such as alopecia areata, vitiligo, ulcerative colitis (UC), rheumatoid arthritis (RA), multiple sclerosis (MS), type 1 diabetes, psoriasis, asthma, and the like, including treatment thereof.

### BACKGROUND OF THE INVENTION

XCL1 and XCL2, also known as lymphotactin-α and -β, respectively, are the sole members of the C chemokine/y chemokine family. XCL1 is expressed and secreted by various immune cells, including activated CD8+ T cells, CD4+ T cells, natural killer (NK) cells, thymic medullary epithelial cells, and others. XCL2 is a paralog of XCL1, and is thought to have arisen from a gene duplication, having a nearly-identical amino acid sequence to XCL1 (differing by only two amino acids in its N-terminal region from XCL1). XCL1 and XCL2 are thought to have similar functions as a result of this structural similarity.

Unlike most other chemokines, which have four cysteine residues near their amino termini capable of forming two disulfide bonds, XCL1 and XCL2 have just two such cysteines, which form a single disulfide bond. This allows the chemokine to be metamorphic, interconverting between a first configuration having a canonical monomeric, α-helix-containing chemokine fold, and a second, primarily β-sheet form. This conversion between forms typically occurs several times per second. The monomeric α-helical form is capable of binding and activating the receptor XCR1 to induce cell migration. The α-helical form undergoes a conformational change to the β-sheet form, which can exist in a monomeric form but favors dimerization. When in the dimeric or β-sheet configuration, XCL1 is inactive for XCR1 binding, and the dimer can adhere to glycosaminoglycan (GAG) on cell surfaces. The monomeric α-helical form does not bind GAG. See Lei and Takahama, Microbes and Infection 14 (2012) 262-267; Fox et al., ACS Chem. Biol. 10 (11) (2015) 2580-2588. The frequent conversion between these forms, particularly given the inactivity of the dimeric and β-sheet form and its tendency to aggregate at the cell surface on GAG, complicates the design of anti-XCL1 and anti-XCL2 antibodies, particularly as therapeutic agents, as an antibody that can bind the dimeric form of the chemokine could primarily bind the inactive form of the chemokine, leading to unknown properties with regard to pharmacokinetics, immunogenicity, and efficacy.

Autoimmune diseases are a form of immune-mediated diseases that arise from the body's production of an immune response against its own tissue. These conditions are often chronic, and can be debilitating and even life-threatening. Current FDA approved treatments for immune-mediated diseases include corticosteroids, often used to treat acute inflammation, and bioproducts targeting TNFα, interleukin-12, -17 and -23. Although these treatments have demonstrated efficacy in reducing symptoms for a subset of patients, a percentage of patients remain nonresponsive or experience a loss of response to the currently available treatments. Thus, there remains a need for additional therapies targeting different human targets and pathways for the treatment of immune-mediated diseases.

A subset of autoimmune diseases has been associated with overactivity of and tissue damage by CD8+ T cells when these cells have migrated to certain sites in the body, including alopecia areata, vitiligo, UC, type 1 diabetes, psoriasis, RA, MS, and asthma, though many of the specifics around the mechanisms of CD8+ T cell activation underlying these conditions have not been elucidated. For example, roles specific to CD8+ T cell activation by dendritic cells (DCs) have generally been elucidated in the field of oncology, rather than immunology, such as in studies of checkpoint antagonists. This is true of the subset of DCs known as conventional dendritic cell 1 (cDC1); see foe example Bowman-Kirigin et al., “The Conventional Dendritic Cell 1 Subset Primes CD8+ T Cells and Traffics Tumor Antigen to Drive Antitumor Immunity in the Brain,” Cancer Immunol Res, 11 (1) (2023) p. 20-37.

While XCL1 antibodies exist as research tools for detection and visualization of XCL1, to date there are no approved or known clinical candidates for XCL1-specific, antagonistic antibody therapeutics. Thus, there remains an unmet need for antibodies, pharmaceutical compositions, and methods which target human XCL1 useful for the treatment of immune-mediated diseases such as RA, MS, asthma, psoriasis, UC, type 1 diabetes, alopecia areata, vitiligo and the like. There is a need for XCL1 antibodies which possess good therapeutic characteristics, including being potent neutralizers (i.e., antagonists) of human XCL1. Advantageous properties of such antibodies may include high binding affinity for human XCL1 and low nonspecific binding, particularly to other chemokines, which may have similar sequence and/or structure to XCL1. Such XCL1 antibodies should also possess therapeutically acceptable pharmacokinetics (Pk) profile and demonstrate low immunogenicity. Such XCL1 antibodies should also be amendable to commercial manufacturing, including high levels of solubility and low levels of aggregation. The present disclosure provides XCL1 antibodies that address one or more of these needs for use in the treatment of immune-mediated diseases.

Accordingly, in certain embodiments, the present invention provides antibodies directed against human XCL1. The antibodies disclosed herein also bind to and neutralize human XCL2.

### SUMMARY OF THE INVENTION

According to some embodiments, the antibodies of the present invention are antagonistic to human XCL1, and in some embodiments, antagonistic to human XCL2. In one aspect, described herein is an antibody that binds human XCL1 which includes a heavy chain variable region (HCVR) and a light chain variable region (LCVR). The HCVR includes complementarity determining regions (CDRs) HCDR1, HCDR2, and HCDR3. The LCVR comprises CDRs LCDR1, LCDR2, and LCDR3. The HCDR1 is or includes SEQ ID NO: 10. The HCDR2 is or includes SEQ ID NO: 11. The HCDR3 is or includes SEQ ID NO: 12, wherein Z is selected from Q and E. The LCDR1 is or includes SEQ ID NO: 5, wherein X is selected from S and D. The LCDR2 is or includes SEQ ID NO: 8. The LCDR3 is or includes SEQ ID NO: 9.

In one embodiment, Z is E. In another embodiment, Z is Q.

In one embodiment, X is D. In another embodiment, X is S.

In one embodiment HCDR1 is or includes SEQ ID NO: 10; HCDR2 is or includes SEQ ID NO: 11; HCDR3 is or includes SEQ ID NO: 13; LCDR1 is or includes SEQ ID NO: 6; LCDR2 is or includes SEQ ID NO: 8; and LCDR3 is or includes SEQ ID NO: 9.

In one embodiment, the LCVR is or includes SEQ ID NO: 17 and the HCVR is or includes SEQ ID NO: 15, wherein Xₐ is selected from Q and pyroglutamic acid. In one embodiment, Xₐ is Q.

In one embodiment, the antibody has a heavy chain (HC) and a light chain (LC), wherein the amino acid sequence of the HC is or includes SEQ ID NO: 19, wherein X₁ is selected from Q and pyroglutamic acid; X₂ is G or absent; and X₃ is K or absent; and the amino acid sequence of the LC is or includes SEQ ID NO: 21. In one embodiment, X₁ is Q; X₂ is G; and X₃ is K.

In one embodiment, HCDR1 is or includes SEQ ID NO: 10; HCDR2 is or includes SEQ ID NO: 11; HCDR3 is or includes SEQ ID NO: 14; LCDR1 is or includes SEQ ID NO: 7; LCDR2 is or includes SEQ ID NO: 8; and LCDR3 is or includes SEQ ID NO: 9.

In one embodiment, the amino acid sequence of the LCVR is or includes SEQ ID NO: 18 and the amino acid sequence of the HCVR is or includes SEQ ID NO: 16, wherein Xₐ is selected from Q and pyroglutamic acid. In one embodiment, Xₐ is Q.

In one embodiment, the antibody has a heavy chain (HC) and a light chain (LC). The HC is or includes SEQ ID NO: 20, wherein X₁ is selected from Q and pyroglutamic acid; X₂ is G or absent; and X₃ is K or absent; and the LC is or includes SEQ ID NO: 22. In one embodiment, X₁ is Q; X₂ is G; and X₃ is K.

In any of the above embodiments, the antibody may be an XCL1 antagonist.

In any of the above embodiments, the antibody may bind human XCL2.

In any of the above embodiments, the antibody may be an XCL2 antagonist.

In any of the above embodiments, the antibody may bind dimeric XCL1. The dimeric XCL1 may be homodimeric XCL1. The antibody may bind XCL1 in a β-sheet configuration. The homodimeric XCL1 may be bound to glycosaminoglycan on a surface of a cell.

In any of the above embodiments, the antibody may bind dimeric XCL2. The dimeric XCL2 may be homodimeric XCL2. The antibody may bind XCL2 in a β-sheet configuration. The homodimeric XCL2 may be bound to glycosaminoglycan on a surface of a cell.

In any of the above embodiments, the antibody may bind a heterodimeric complex including one monomer of XCL1 and one monomer of XCL2. In such an embodiment, the antibody binds at least one amino acid of the XCL1 monomer or at least one amino acid of the XCL2 monomer of the heterodimeric complex. The heterodimeric complex may be bound to glycosaminoglycan on a surface of a cell.

In any of the above embodiments, the antibody does not bind a chemokine other than XCL1 and XCL2.

In one embodiment, the antibody includes a heavy chain variable region (HCVR) having at least 95% sequence identity to SEQ ID NO: 15 and a light chain variable region (LCVR) having at least 95% sequence identity to SEQ ID NO: 17.

In one embodiment, the antibody includes a heavy chain (HC) having at least 95% sequence identity to SEQ ID NO: 19, and a light chain (LC) having at least 95% sequence identity to SEQ ID NO: 21.

In one embodiment, the antibody includes a heavy chain variable region (HCVR) having at least 95% sequence identity to SEQ ID NO: 16 and a light chain variable region (LCVR) having at least 95% sequence identity to SEQ ID NO: 18.

In one embodiment, the antibody includes a heavy chain (HC) having at least 95% sequence identity to SEQ ID NO: 20, and a light chain (LC) having at least 95% sequence identity to SEQ ID NO: 22.

In one embodiment, the antibody binds an epitope of XCL1 including one or more amino acids of SEQ ID NO: 27. In one embodiment, the antibody binds two or more amino acids of SEQ ID NO: 27. In an embodiment, the antibody binds three or more amino acids of SEQ ID NO: 27.

In one embodiment, the antibody binds one or more amino acids of SEQ ID NO: 29.

In one embodiment, the antibody binds an epitope of XCL1 including one or more amino acids of SEQ ID NO: 28. In one embodiment, the antibody binds two or more amino acids of SEQ ID NO: 28.

In embodiment, the antibody which binds an epitope of XCL1 including one or more amino acids of SEQ ID NO: 27 and one or more amino acids of SEQ ID NO: 28. In one embodiment, the antibody binds two or more amino acids of SEQ ID NO: 27 and to or more amino acids of SEQ ID NO: 28. In one embodiment, the antibody binds one or more amino acids of SEQ ID NO: 29. In one embodiment, the antibody has a human IgG1 isotype.

The present disclosure also provides a pharmaceutical composition including an antibody as described in any of the above embodiments and one or more pharmaceutically acceptable carriers, diluents or excipients.

The present disclosure provides a method of treating alopecia areata, vitiligo, asthma, psoriasis, multiple sclerosis, psoriasis, rheumatoid arthritis, or ulcerative colitis in a subject in need thereof, comprising administering to the subject an effective amount of an antibody or a pharmaceutical composition as described herein.

The present disclosure provides an antibody or pharmaceutical composition for use in therapy, such as for use in the treatment of alopecia areata, vitiligo, asthma, psoriasis, multiple sclerosis, rheumatoid arthritis, or ulcerative colitis.

The present disclosure provides for the use of an antibody or a pharmaceutical composition in the manufacture of a medicament for the treatment of alopecia areata, vitiligo, asthma, psoriasis, multiple sclerosis, rheumatoid arthritis, or ulcerative colitis.

In one aspect, a nucleic acid including a sequence encoding SEQ ID NO: 23, SEQ ID NO: 24, SEQ ID NO: 25, or SEQ ID NO: 26, is provided. In one aspect, a vector including a first nucleic acid sequence encoding SEQ ID NO: 23 or SEQ ID NO: 25 and a second nucleic acid sequence encoding SEQ ID NO: 24 or SEQ ID NO: 26 is described, as is a cell including such a vector. In one embodiment, a method of producing an antibody including culturing said cell under conditions such that the antibody is expressed and recovering the expressed antibody from the culture medium is disclosed. In one embodiment, an antibody produced by culturing said cell under conditions such that the antibody is expressed and recovering the expressed antibody from the culture medium is disclosed.

In one aspect, a protein complex comprising dimeric XCL1 and an antibody bound to dimeric XCL1 is provided. The dimeric XCL1 may be homodimeric XCL1. The antibody may bind at least one amino acid residue on each monomeric unit of dimeric XCL1.

In one aspect, a protein complex comprising dimeric XCL2 and an antibody bound to dimeric XCL2 is provided. The dimeric XCL2 may be homodimeric XCL2. The antibody may bind at least one amino acid residue on each monomeric unit of dimeric XCL2.

In one aspect, a protein complex including a heterodimer made up of a monomeric unit of XCL1 and a monomeric unit of XCL2, and an antibody bound to the heterodimer, is provided. In this embodiment, the antibody may bind at least one amino acid residue of XCL1, or at least one amino acid residue of XCL2, or at least one residue of XCL1 and at least one residue of XCL2.

### DETAILED DESCRIPTION OF THE INVENTION

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)₂, Fv fragments, scFv antibody fragments, scFab, disulfide-linked Fvs (sdFv), a Fd fragment.

The antibodies of the present invention are monoclonal antibodies. Monoclonal antibodies are antibodies derived from a single copy or clone including, for example, any eukaryotic, prokaryotic or phage clone, and not the method by which it is produced. Monoclonal antibodies can be produced, for example, by hybridoma technologies, recombinant technologies, phage display technologies, synthetic technologies, e.g., CDR-grafting, or combinations of such or other technologies known in the art.

Methods of producing and purifying antibodies are well known in the art and can be found, for example, in Harlow and Lane (1988), Antibodies, A Laboratory Manual, Cold Spring Harbor Laboratory Press, Cold Spring harbor, N.Y., chapters 5-8 and 15, ISBN 0-87969-314-2. For example, mice or rabbits including transgenic mice or rabbits as known in the art, may be immunized with human XCL1, or portions thereof, and the resulting antibodies can be recovered, screened, purified, and the amino acid sequences determined using conventional methods well known in the art.

In particular embodiments of the present invention, the antibody, or the nucleic acid encoding same, is provided in isolated form. As used herein, the term “isolated” refers to a protein, peptide, or nucleic acid which is free or substantially free from other macromolecular species found in a cellular environment.

The antibodies of the present invention may be prepared and purified using known methods. For example, cDNA sequences encoding a HC (for example the amino acid sequence given by SEQ ID NO: 19 or SEQ ID NO: 20) and a LC (for example, the amino acid sequence given by SEQ ID NO: 21 or SEQ ID NO: 22) may be cloned and engineered into a GS (glutamine synthetase) expression vector. In one example, an antibody has a heavy chain with amino acid sequence given by SEQ ID NO: 19 and a light chain with amino acid sequence given by SEQ ID NO: 21; in such an instance, cDNA sequences which may be used to express the chains are provided by SEQ ID NO: 23 and SEQ ID NO: 24, respectively. In another example, an antibody has a heavy chain with amino acid sequence given by SEQ ID NO: 20 and a light chain with amino acid sequence given by SEQ ID NO: 22; in this instance, cDNA sequences which may be used to express the chains are provided by SEQ ID NO: 25 and SEQ ID NO: 26, respectively.

The engineered immunoglobulin expression vector may then be stably transfected into CHO cells. As one of skill in the art will appreciate, mammalian expression of antibodies will result in glycosylation, typically at highly conserved N-glycosylation sites in the Fc region. Stable clones may be verified for expression of an antibody specifically binding to human XCL1 (for example, as represented by a recombinantly produced peptide comprising SEQ ID NO: 1). Positive clones may be expanded into serum-free culture medium for antibody production in bioreactors. Media, into which an antibody has been secreted, may be purified by conventional techniques. For example, the medium may be conveniently applied to a Protein A or G Sepharose FF column that has been equilibrated with a compatible buffer, such as phosphate buffered saline. The column is washed to remove nonspecific binding components. The bound antibody is eluted, for example, by pH gradient and antibody fractions are detected, such as by SDS-PAGE, and then pooled. The antibody may be concentrated and/or sterile filtered using common techniques. Soluble aggregate and multimers may be effectively removed by common techniques, including size exclusion, hydrophobic interaction, ion exchange, or hydroxyapatite chromatography. The product may be immediately frozen, for example at −70° C., or may be lyophilized.

The antibodies of the present invention can be used in the treatment of patients. More particularly the antibodies of the present invention are expected to be useful in treating immune-mediated diseases or disorders, which include alopecia areata, vitiligo, asthma, RA, MS, UC, psoriasis, and type 1 diabetes. As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of an antibody, or pharmaceutical composition thereof, of the present invention for treatment of a disease or condition in a human that would benefit from a reduction in XCL1 and/or XCL2 activity, and includes: (a) inhibiting further progression of the disease, i.e., arresting its development; or (b) relieving the disease, i.e., causing regression of the disease or disorder, or alleviating symptoms or complications thereof.

As used interchangeably herein, the term “patient,” “subject,” and “individual,” refers to a human. In certain embodiments, the patient is further characterized with a disease, disorder, or condition (e.g., an immune-mediated disease) that would benefit from a reduction in XCL1 and/or XCL2 activity. In other embodiments, the patient is further characterized as being at risk of developing an immune-mediated disease, disorder, or condition that would benefit from a reduction in XCL1 and/or XCL2 activity.

As referred to herein, the term “epitope” refers to the amino acid residues, of an antigen, that are bound by an antibody. An epitope can be a linear epitope, a conformational epitope, or a hybrid epitope.

The term “epitope” may be used in reference to a structural epitope. A structural epitope, according to some embodiments, may be used to describe the region of an antigen which is covered by an antibody (e.g., an antibody's footprint when bound to the antigen). In some embodiments, a structural epitope may describe the amino acid residues of the antigen that are within a specified proximity (e.g., within a specified number of Angstroms) of an amino acid residue of the antibody.

The term “epitope” may also be used in reference to a functional epitope. A functional epitope, according to some embodiments, may be used to describe amino acid residues of the antigen that interact with amino acid residues of the antibody in a manner contributing to the binding energy between the antigen and the antibody.

An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3^{rd} ed. 2018), including but not limited to, X-ray crystallography, nuclear magnetic resonance (NMR) spectroscopy, site-directed mutagenesis, species swap mutagenesis, alanine-scanning mutagenesis, hydrogen-deuterium exchange (HDX) and cross-blocking assays.

The terms “bind,” “binds,” and “binding” as used herein are intended to mean, unless indicated otherwise, the ability of a protein or molecule to form a chemical bond or attractive interaction with another protein or molecule, which results in proximity of the two proteins or molecules as determined by common methods known in the art. With particular reference to binding of antibody to antigen, the terms “bind,” “binds,” and “binding” refer to the interaction of one or more of the CDRs of the hypervariable region of the antibody with the antigen; that is, specific binding may be conferred to the antibody by one or more of the CDRs.

The terms “specifically binds” and “specific binding” as used herein refer to the situation in which one member of a specific binding pair does not significantly bind to molecules other than its specific binding partner(s). For instance, certain antibodies may be said to specifically bind to XCL1 and XCL2, but not other similar proteins (such as chemokines, etc.) An antibody may be said to bind specifically to an antigen if it binds at least about 50% greater, or 2-fold greater, or 20-fold greater, or 50-fold greater, or 100-fold greater than it binds a different antigen or non-antigen target as measured by a technique available in the art, e.g., competition ELISA or K_{D} measurement with a BIACORE or KINEXA assay.

An antibody of the present invention can be incorporated into a pharmaceutical composition which can be prepared by methods well known in the art and comprise an antibody of the present invention and one or more pharmaceutically acceptable carrier(s) and/or diluent(s) (e.g., Remington, The Science and Practice of Pharmacy, 22^{nd} Edition, Loyd V., Ed., Pharmaceutical Press, 2012, which provides a compendium of formulation techniques as are generally known to practitioners). Suitable carriers for pharmaceutical compositions include any material which, when combined with an antibody of the present invention, retains the molecule's activity and is non-reactive with the patient's immune system.

A pharmaceutical composition comprising an antibody of the present invention can be administered to a patient at risk for, or exhibiting, diseases or disorders as described herein by parental routes (e.g., subcutaneous, intravenous, intraperitoneal, intramuscular, or transdermal). A pharmaceutical composition of the present invention contains an “effective” or “therapeutically effective” amount, as used interchangeably herein, of an antibody of the present invention. An effective amount refers to an amount necessary (at dosages and for periods of time and for the means of administration) to achieve the desired therapeutic result. An effective amount of an antibody may vary according to factors such as the disease state, age, sex, and weight of the individual, and the ability of the antibody to elicit a desired response in the individual. An effective amount is also one in which any toxic or detrimental effects of the antibody of the present invention are outweighed by the therapeutically beneficial effects.

Percent homology, as used in the present disclosure, in the context of two or more amino acid sequence refers to two or more sequences having a specified percentage of amino acid residues that are the same, when compared and aligned for maximum correspondence, as measured using a sequence comparison algorithm (e.g., BLASTP and BLASTN or other algorithms available to persons of skill) or by visual inspection. Depending on the application, the percent homology can exist over a region of the sequence being compared, e.g., over a functional domain, or, alternatively, exist over the full length of the two sequences to be compared. By way of example, percent homology of a sequence may be compared to a reference sequence. For example, when using a sequence comparison algorithm, test and reference sequences may be input into a computer (and subsequence coordinates may be further designated if desired along with sequence algorithm program parameters). The sequence comparison algorithm then calculates the percent sequence identity or homology for the test sequence(s) relative to the reference sequence(s), based on the designated program parameters. Exemplary sequence alignment and/or homology algorithms are available through, Smith & Waterman, Adv. Appl. Math. 2:482 (1981), Needleman & Wunsch, J. Mol. Biol. 48:443 (1970), Pearson & Lipman, Proc. Nat'l. Acad. Sci. USA 85:2444 (1988), GAP, BESTFIT, FASTA, and TFASTA (in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

It is known that in some cases, recombinant production of antibodies can lead to modifications of the antibody. Examples of such modifications include loss of a lysine or glycine at the C-terminus of the HC; conversion of a glutamine or glutamic acid at the N-terminus of the heavy chain to pyroglutamic acid; or conversion of a glutamine or glutamic acid at the N-terminus of a lambda light chain to pyroglutamic acid. This may result in production a heterogeneous mix of antibodies, including those with no modification, a single modification, or multiple modifications. The antibodies of the present disclosure may undergo such modifications and are encompassed by the claims derived therefrom.

In one embodiment, the antibody of the present invention binds XCL1 and neutralizes XCL1. In one embodiment, the antibody of the present invention binds XCL2 and neutralizes XCL2.

In one embodiment, the antibody of the present invention impedes or prevents the migration of a cell expressing XCR1. In a specific embodiment, the antibody of the present invention neutralizes dendritic cell migration. In one embodiment, the dendritic cell is a cDC1.

In one embodiment, the antibody of the present invention has a K_{D} for XCL1 of less than 5.0×10⁻¹¹ M. In one embodiment, the antibody of the present invention has a K_{D} for XCL2 of less than 1.0×10⁻¹¹ M.

In one embodiment, the antibody of the present invention binds to XCL1 in its β-sheet monomer configuration. In one embodiment, the antibody of the present invention binds to XCL1 in its dimeric configuration.

In one embodiment, the antibody of the present invention binds to XCL2 in its β-sheet monomer configuration. In one embodiment, the antibody of the present invention binds to XCL2 in its dimeric configuration.

In one embodiment, the antibodies disclosed herein bind homodimeric XCL1. In an embodiment, the antibodies disclosed herein bind homodimeric XCL2. In one embodiment, the antibodies herein bind heterodimeric complexes which include one monomer of XCL1 and one monomer of XCL2. In one embodiment, the antibodies disclosed herein bind to free monomeric XCL1 and/or XCL2, and/or free homodimeric XCL1, and/or free homodimeric XCL2, and/or free heterodimers of XCL1 and XCL2. In one embodiment, the antibodies of the present disclosure bind to homodimeric XCL1 bound to GAGs, and/or homodimeric XCL2 bound to GAGs, and/or heterodimers of XCL1 and XCL2 bound to GAGs.

In one embodiment, antibody binds a sequence given by SEQ ID NO: 29 (that is, threonine 16, glutamine 17, arginine 18, leucine 19, and proline 20). In an embodiment, the antibody binds sequence given by SEQ ID NO: 29 along with lysine 42 and arginine 43. In one embodiment, an antibody of the present invention binds one or more amino acids of SEQ ID NO: 27; or two or more amino acids of SEQ ID NO: 27; or three or more amino acids of SEQ ID NO: 27. In one embodiment, an antibody of the present invention binds one or more amino acids of SEQ ID NO: 28; or two or more amino acids of SEQ ID NO: 28. In an embodiment, an antibody of the present invention binds one or more amino acids of SEQ ID NO: 29. In one embodiment, an antibody of the present invention binds one or more amino acids of SEQ ID NO: 27 and one or more amino acids of SEQ ID NO: 28; or two or more amino acids of SEQ ID NO: 27 and two or more amino acids of SEQ ID NO: 28.

In one embodiment, an antibody of the present invention has anti-inflammatory activity, and may be used as a therapeutic agent.

In one aspect, the antibody of the present invention binds to XCL1 and/or XCL2, but does not specifically bind to another chemokine.

### EXAMPLES

### Example 1: Expression of Recombinant XCL1 and Variants Thereof, and XCL2

Each protein (human XCL1, SEQ ID NO: 1; human XCL2, SEQ ID NO: 2; CC3, SEQ ID NO: 3; and CC5, SEQ ID NO: 4) was expressed as a fusion protein structured with an 8×His tag at the N-terminus, the well-known BRIL fusion protein, an enterokinase (EK) cleavage site, and then the specific protein (XCL1, etc.) at the C-terminal end. A fusion protein expression plasmid was used to create a stable CHO-K1 cell line for each protein by methods known in the art. The fusion proteins were expressed and secreted in chemically defined media. The media was harvested, and fusion proteins were captured on a Ni-IMAC column (for example, as is available from Pierce) and eluted with 375 mM imidazole at pH 7.5. The Ni-IMAC pool was dialyzed into the EK digestion buffer (20 mM Tris, 100 mM NaCl, 5 mM CaCl₂); pH 8), and the fusion protein was incubated overnight with EK. The free protein was captured with Capto-S resin (Cytiva Cat #17544101) before polishing by size exclusion chromatography (Superdex 75—Cytiva Cat #28989333). Protein identity was confirmed by mass spectrometric analysis.

### Example 2: Expression of Anti-XCL1 Antibodies

Exemplified anti-XCL1 antibodies of the present invention are presented in Table 1 below. Antibodies were produced in stable CHO-K1 cell lines in a chemically defined media. They were captured on PRISMA resin (Cytiva—Cat #1754981) and eluted with a pH 3 buffer. PRISMA pool was brought to pH 5 to precipitate host cell proteins (HCP). The proteins were polished by anion exchange chromatography (AEX) using a POROS 50 HS (Thermo Fisher Cat #1335906) and then dialyzed into 10 mM Histidine, 275 mM Sucrose; pH 6. Material was analyzed by analytical size exclusion chromatography (SEC) and confirmed using mass spectrometry.

TABLE 1
SEQ ID Nos of Exemplified Antibody Amino Acid Sequences

| | HC | HCVR | HCDR1 | HCDR2 | HCDR3 | LC | LCVR | LCDR1 | LCDR2 | LCDR3 |
|---|---|---|---|---|---|---|---|---|---|---|
| Ab 1 | 19 | 15 | 10 | 11 | 13 | 21 | 17 | 6 | 8 | 9 |
| Ab 2 | 20 | 16 | 10 | 11 | 14 | 22 | 18 | 7 | 8 | 9 |

A control antibody specifically binding to monomeric XCL1 (that is, which binds CC3 with high affinity and does not bind CC5; see below Examples), with HC amino acid sequence given by SEQ ID NO: 27 and LC amino acid sequence given by SEQ ID NO: 28. cDNA sequences which may be used for expressing the HC and LC are given by SEQ ID NO: 29 and SEQ ID NO: 30, respectively. This antibody is referred to herein as monomer-binding control (MBC).

The relationship of the various regions of exemplified anti-XCL1 antibodies is as follows (numbering of amino acids applies linear numbering; assignment of amino acids to variable domains is based on the International Immunogenetics Information System® available at www.imgt.org.) Assignment of amino acids to CDR domains for each antibody exemplified is based on the well-known North numbering convention, with the exception of HCDR2, which C-terminus is based on the well-known Kabat numbering convention.

Exemplified antibodies of the present disclosure are identified as possessing high binding affinity and being chemically and physically stabile including aggregation and solubility consistent with therapeutic parental administration. The exemplified antibodies of the present disclosure are also identified as possessing low immunogenicity (including low nonspecific and/or serum binding) and possessing pharmacokinetic properties consistent with therapeutic parental administration for the treatment of immune-mediated diseases. In one aspect, the antibodies disclosed herein are human antibodies of the IgG1 isotype. The antibodies disclosed herein are monoclonal antibodies. Antibodies of other isotypes (for example, IgG4, and so forth) are also contemplated by this disclosure.

### Example 3: Properties of XCL1 Variants CC3 and CC5

XCL1 is a metamorphic protein that readily undergoes major conformational rearrangements depending on salt and temperature conditions. High salt and low temperature (10° C.) favors an active monomer conformation having an α-helix motif typical of canonical chemokine folds that can signal through the cognate receptor, XCR1, while low salt and higher temperature (40° C.) favors an inactive β-sheet configuration which can dimerize and bind to endogenous glycosaminoglycans (GAGs).

To study the properties of each configuration separately, engineered variant proteins were expressed and purified. CC3 (SEQ ID NO: 3) is an engineered variant of XCL1 stabilized in the active, α-helix-containing monomer conformation through creation of a disulfide bond when residues 21 and 59 are mutated to cysteine (V21C/V59C). Likewise, mutations to cysteine at residues 36 and 49 (A36C/A49C) create an alternative disulfide structure that stabilizes the inactive β-sheet configuration which tends toward the dimeric state, termed CC5 (SEQ ID NO: 4). These variants are known in the art and were prepared as specified in the literature (see Biopolymers 2021 October; 112 (10): e23402 doi: 10.1002/bip.23402. Epub 2020 Sep. 28.)

XCR1 is expressed on several types of dendritic cells (DC), primarily type 1 conventional dendritic cells (cDC1). cDC1 are antigen presenting cells (APC) adept at antigen presentation to CD8+ T cells in the context of major histocompatibility complex I. As CD8+ T cells secrete XCL1, a chemotactic gradient is generated, attracting XCR1-bearing cells to the site of the CD8+ T cells (for instance, within a tumor or autoimmune site), augmenting the immune response. See Dorner et al., Immunity 31 (2009) 823-833; Audsley et al. Cells 9 (2020) 565 1-22.

Recombinant human XCL1, CC3, and CC5 were prepared and evaluated in a migration assay utilizing a murine Ba/F3 cell line stably overexpressing human XCR1. This cell line migrates toward an XCL1 chemokine gradient in vitro, in a 5 μM Boyden Chamber assay plate. The assay is set up with 150,000 cells/well in the top chamber with various concentrations of XCL1 added to the bottom chamber. Cells are permitted to migrate over approximately 4 hours at 37° C., with the translocated cells quantitated using a luminescent cell viability readout. Results are shown below in Table 2.

TABLE 2
Migration of XCR1-expressing cells
in the presence of XCL1 variants

| | | Error | |
|---|---|---|---|
| | GeoMean | (delta | |
| Ligand | EC₅₀ (nM) | method) | n |
| Human XCL1 | 858.4 | 82.0 | 3 |
| wild-type | | | |
| Human XCL1 | 114.7 | 65.4 | 3 |
| CC3 | | | |
| Human XCL1 | Inactive | — | 3 |
| CC5 | | | |

For CC3, robust migration occurs with an EC₅₀ around 114.7 nM compared to wild-type XCL1 (858.4 nM). For CC5, migration was not observed. These data confirm observations in the literature indicating that recombinant CC3 and CC5 represent the two distinct conformations of XCL1.

### Example 4: Binding Kinetics and Affinity

The affinity and binding kinetics of antibodies of the present invention and the MBC to human XCL1, human XCL2, mouse XCL1, cynomolgus (cyno) XCL1, and rat XCL1 were determined by surface plasmon resonance using Biacore 8K (GE Healthcare). The procedure generally follows the “Instrument Handbook.” Antibody was first captured on a Biacore Protein A chip followed by flowing a serial concentration of antigens from 400 nM down to 0.039 nM in 2-fold serial dilution in PBS-P20-BSA (0.1% surfactant P20, 0.1 mg/mL BSA). All measurements are carried out at 25° C.

The multi-cycle kinetics setting that runs each analyte concentration in a separate cycle regenerating the surface after each sample injection is used. The regeneration is optimized to maintain consistent surface properties from cycle to cycle. Each cycle starts with a 3 minute injection of antibody (approximately 150 response units (RU) capture level) at 10 μl/min flow rate, followed by 3 minute injection of antigen at 50 ul/min flow rate and a 15 min dissociation phase in PBS-P20-BSA. The chip surface was then regenerated with 30 seconds injection of pH 1.5, 10 mM glycine buffer at 50 μl/min flow rate for three times. The data was fit to a 1:1 binding model to derive kₐ and k_{d}, and to calculate K_{d}.

Following procedures essentially as described above, the following parameters are obtained for the antibodies in the present invention.

TABLE 3
Antibody affinity and kinetics with various XCL1 antigens

| | | huXCL1 | | | msXCL1 | | |
|---|---|---|---|---|---|---|---|
| | | kₐ | k_{d} | K_{d} | kₐ | k_{d} | K_{d} |
| Antibody | | (1/Ms) | (1/s) | (M) | (1/Ms) | (1/s) | (M) |
| 1 | | 5.0E+07 | 6.7E−04 | 1.3E−11 | 1.1E+06 | 1.4E−04 | 1.3E−10 |
| 2 | | 9.2E+06 | 2.9E−04 | 3.2E−11 | 6.1E+05 | 2.1E−04 | 3.5E−10 |
| MBC | | 3.5E+07 | 8.2E−05 | 2.4E−12 | 1.9E+05 | 1.3E−04 | 6.8E−10 |
| | | cynoXCL1 | | | Rat XCL1 | | |
| | | kₐ | k_{d} | K_{d} | kₐ | k_{d} | K_{d} |
| | Antibody | (1/Ms) | (1/s) | (M) | (1/Ms) | (1/s) | (M) |
| | 1 | 1.5E+08 | 5.7E−04 | 3.9E−12 | 9.6E+06 | 6.1E−05 | 6.3E−12 |
| | 2 | 2.5E+07 | 2.8E−04 | 1.1E−11 | 4.5E+06 | 1.5E−04 | 3.2E−11 |
| | MBC | 2.2E+07 | 2.9E−04 | 1.3E−11 | 4.0E+06 | <1e−6 | <1e−12 |

| | huXCL2 | | | CC3 | | | CC5 | | |
|---|---|---|---|---|---|---|---|---|---|
| | kₐ | k_{d} | K_{d} | kₐ | k_{d} | K_{d} | kₐ | k_{d} | K_{d} |
| Antibody | (1/Ms) | (1/s) | (M) | (1/Ms) | (1/s) | (M) | (1/Ms) | (1/s) | (M) |

| | | | | | | | |
|---|---|---|---|---|---|---|---|
| 1 | 1.8E+07 | 6.4E−05 | 3.5E−12 | No Binding | 8.3E+06 | 2.1E−04 | 2.6E−11 |
| 2 | 6.8E+06 | 5.2E−05 | 7.7E−12 | No Binding | 8.2E+06 | 1.8E−04 | 2.2E−11 |

| | | | | | | | |
|---|---|---|---|---|---|---|---|
| MBC | 2.9E+06 | 1.0E−05 | 3.5E−12 | 1.1E+05 | 2.1E−04 | 1.9E−09 | No Binding |

Notably, Antibodies 1 and 2 bind CC5 with good affinity but not CC3, whereas the opposite is observed in the monomer-specific binding of MBC. This may be because the introduction of cysteines in CC3 may interfere with the epitope to which Antibodies 1 and 2 bind. Crystallography experiments support Antibody 1 and Antibody 2 binding to XCL1 in β-sheet monomer form. In one aspect, Antibodies 1 and 2 can be referred to as primarily and preferentially binding the dimeric form, and these results demonstrate that Antibodies 1 and 2 can bind to homodimeric XCL1.

### Example 5: Neutralization of XCL1 and XCL2: Human XCR1 Migration Assay

XCL1 and XCL2 are highly conserved chemokines with chemotactic activity mediated through the XCR1 receptor. The murine cell line Ba/F3 was stably transfected with human XCR1 receptor to assess the ability of XCL1/2 to induce migration in an in vitro system.

For this assay, 150,000 recombinant hXCR1/Ba/F3 cells were seeded in the top chamber of a 5 μM Boyden Chamber assay plate with XCL1 concentrations empirically determined for each species added to the bottom chamber. The cells were allowed to migrate toward the XCL1/2 gradient for approximately 4 hours with the cells in the bottom chamber assayed by luminescent cell viability readout. In this assay, human XCL1, human XCL2, mouse, rat and cyno XCL1 all induce migration. After a dose-response of ligand was conducted, a single concentration of XCL1/2 was selected and used into the bottom chamber of the Boyden Chamber plate along with a dose-response of each mAB tested. Neutralization IC₅₀ values for each antibody were determined. As seen in Table 4 below, all antibodies were able to neutralize human XCL1 and XCL2, as well as rat, mouse and cyno XCL1.

TABLE 4
Neutralization of XCL1 and XCL2 by antibodies of the present invention

| Ligand | | | GeoMean | Error | | | | |
|---|---|---|---|---|---|---|---|---|
| (XCL1 if not | Ligand | | IC₅₀ | (delta | | Log | | N |
| specified) | (nM) | Antibody | (nM) | method) | n | Mean | sd | Day |
| Rat | 7.79 | 1 | 2.56 | 0.25 | 3 | 0.41 | 0.07 | 3 |
| Rat | 7.79 | 2 | 5.65 | 0.29 | 3 | 0.75 | 0.04 | 3 |
| Mouse | 0.49 | 1 | 1.07 | 0.22 | 3 | 0.03 | 0.15 | 3 |
| Mouse | 0.49 | 2 | 2.98 | 0.90 | 3 | 0.47 | 0.23 | 3 |
| Cyno | 7.79 | 1 | 4.07 | 0.22 | 3 | 0.61 | 0.04 | 3 |
| Cyno | 7.79 | 2 | 4.51 | 0.16 | 3 | 0.65 | 0.03 | 3 |
| human XCL2 | 7.79 | 1 | 2.30 | 0.01 | 3 | 0.36 | 0.00 | 3 |
| human XCL2 | 7.79 | 2 | 2.53 | 0.21 | 3 | 0.40 | 0.06 | 3 |
| human XCL1 | 2.43 | 1 | 0.60 | 0.05 | 3 | −0.22 | 0.06 | 3 |
| human XCL1 | 2.43 | 2 | 0.66 | 0.10 | 3 | −0.18 | 0.12 | 3 |

The results provided in Tables 3 and 4 demonstrate that both exemplified antibodies bind both XCL1 and XCL2 with high affinity, and neutralize both chemokines in in vitro assays. Therefore it will be appreciated that the antibodies of the present disclosure are useful for binding and neutralization of XCL1, XCL2, or both, including in a therapeutic context. It will be appreciated that the antibodies as disclosed herein bind homodimeric XCL1, and that because of the sequence similarity between XCL1 and XCL2, that the antibodies bind homodimeric XCL2. The antibodies will also bind heterodimeric complexes which may form and which include one monomer of XCL1 and XCL2. The antibodies of the present disclosure may bind to free monomeric XCL1 or XCL2, or free homodimeric XCL1, or free homodimeric XCL2, or free heterodimers of XCL1 and XCL2. The antibodies of the present disclosure may also bind to homodimeric XCL1 bound to GAGs, or homodimeric XCL2 bound to GAGs, or heterodimers of XCL1 and XCL2 bound to GAGs.

### Example 6: Neutralization of XCL1 Activity by Antibodies: Dendritic Cell Migration

XCL1 and XCL2 stimulate migration of XCR1 expressing conventional type 1 dendritic cells (cDC1). To demonstrate XCL1/2 antibody neutralization activity, human cDC1 cells were differentiated from CD34+ stem cells and their migration towards XCL1 through a Boyden Chamber was measured by flow cytometry. Briefly, CD34+ cells from human cord blood were differentiated over 3 weeks in a cytokine cocktail on a feeder layer expressing DLL1 (adapted from Balan et al. doi: 10.1016/j.celrep.2018.07.033). Cells were enriched using a myeloid dendritic cell kit (StemCell; lineage negative, HLA-DR⁺, CD11c⁺). 10 ng/ml XCL1+antibody dose titrations in triplicate were pre-incubated 1 hour and added to the bottom chamber of the Boyden Chamber. Antibodies employed were Antibody 1, Antibody 2, and hIgG1-AAS isotype control antibody. 150,000 enriched, differentiated cells were added to the top chamber of the Boyden Chamber and incubated for 2 hours. Migrated human cDC1 cells in the bottom chamber were stained (live/dead, CLEC9A⁺, XCR1⁺) and counted. The present XCL1/2 antibodies completely inhibited cDC1 migration towards XCL1 across 3 replicate experiments and the GeoMean of IC₅₀ values (nM) and associated error (delta method) are shown in Table 5.

TABLE 5
Potency of XCL1/2 antibody inhibition of XCL1
stimulated primary human cDC1 migration

| Antibody | GeoMean IC₅₀ (nM) | Error (delta method) |
|---|---|---|
| 1 | 0.194 | 0.044 |
| 2 | 0.363 | 0.060 |
| Control | No inhibition | — |

### Example 7: Anti-Inflammatory Effects of Antibody in Mouse

The immunosuppressive activities of Antibodies 1 and 2 were assessed in a mouse model of oxazolone-induced delayed-type hypersensitivity on mouse ears and compared to an anti-CD8 depleting antibody (Bio X Cell #BE0117). Mice were treated with anti-XCL1/2 antibodies on day 0, approximately 1 hour prior to sensitization with 3% oxazolone in acetone on their shaved, ventral-side surface. On day 6, mice were challenged with 2% oxazolone administration on the ear with change in ear swelling measured on day 7. Results are shown in Table 6, as delta ear thickness with anti-XCL1/2 antibodies compared to a matched isotype.

TABLE 6
Anti-inflammatory action in mouse model

| | GeoMean (delta | | |
|---|---|---|---|
| | thickness (mm) | | |
| | compared to | | |
| Antibody | isotype (%)) | Error | n |
| 1 | −40 | 3.5 | 2 |
| 2 | −43 | 1.0 | 2 |
| Anti-CD8 | −38 | 4.5 | 2 |

### Example 8: Epitope Mapping and Determination

Binding sites for the exemplified antibodies on the surface of XCL1 were determined by HDX and X-ray crystallography, techniques well known in the art. By HDX, Antibodies 1 and 2 were found to bind a non-linear epitope formed of residues within residues 12 to 20 (SEQ ID NO: 27), and within residues 35 to 46, SEQ ID NO: 28. An X-ray crystal structure of a fragment antigen-binding region (Fab) complexed with human XCL1, to 2.77 angstrom resolution, further suggests that the specific residues involved in binding are those whose sequence is given by SEQ ID NO: 29 (that is, threonine 16, glutamine 17, arginine 18, leucine 19, and proline 20), along with lysine 42 and arginine 43. An antibody of the present invention may therefore bind one or more amino acids of SEQ ID NO: 27; or two or more amino acids of SEQ ID NO: 27; or three or more amino acids of SEQ ID NO: 27. An antibody of the present invention may bind one or more amino acids of SEQ ID NO: 28; or two or more amino acids of SEQ ID NO: 28. An antibody of the present invention may bind one or more amino acids of SEQ ID NO: 29. An antibody of the present invention may bind one or more amino acids of SEQ ID NO: 27 and one or more amino acids of SEQ ID NO: 28; or two or more amino acids of SEQ ID NO: 27 and two or more amino acids of SEQ ID NO: 28.

The antibodies exemplified herein bind to a non-linear epitope of XCL1/XCL2. In one instance, the antibodies bind to a monomeric β-sheet configuration of XCL1 or XCL2. In another instance, the antibodies bind to a dimeric form of XCL1 or XCL2. In such a case, the antibody may bind to a single one of the two monomer subunits of chemokine which come together to define the dimer. For example, the antibody may bind to amino acid residues defined by SEQ ID NO: 29 and lysine 42 and/or arginine 43 of a first monomeric unit of XCL1. This disclosure likewise contemplates an antibody which binds dimeric XCL1 or XCL2 by binding a non-linear epitope which includes residues of both monomers of the dimer. Another binding mode contemplated herein is that of binding monomeric β-sheet XCL1 or XCL2 in such a way that dimerization is prevented.

### Example 9: GAG-Binding Assay

As mentioned, the β-sheet configuration of XCL1/2 is able to dimerize and cannot bind the XCR1 receptor. Instead, in this form XCL1/2 binds to cell surface GAGs, which is thought to help establish a chemoattractive gradient. Antibodies 1 and 2 bind to the β-sheet configuration, preferentially to the dimer form, and their ability to bind to XCL1/2 dimers bound to cell surface GAGs using flow cytometry was studied.

293T cells were incubated with 1 μg/mL of various preparations of XCL1 or XCL2, and 100 nM of either Antibody 1, Antibody 2, or hIgG1-AAS isotype control antibody (“Control”) for 1 hour. This was followed by staining with anti-human-Fc/PE secondary antibody (Life Technologies 12-4998-82) to detect the antibody-XCL1/2 complex bound to cell surface GAG. In another condition, the binding of antibody-XCL1/2 complex is done in the presence of 100 μg/mL heparin to demonstrate the specificity of GAG binding. The mean fluorescence intensity (MFI) was measured by flow cytometry for each condition. The experiment was repeated 3 times and the average MFI+/−standard deviation across experiments is shown below in Table 7.

TABLE 7
Detection of XCL1/2 antibody binding to 293T cell
surface XCL1/2-GAG by flow cytometry (MFI)

| | Human XCL1 | | Cyno XCL1 | | Rat XCL1 | |
|---|---|---|---|---|---|---|
| Antibody | MFI | +/− | MFI | +/− | MFI | +/− |
| 1 | 213.7 | 136.1 | 95.3 | 27.0 | 112.1 | 19.1 |
| 1 + | 25.9 | 9.8 | 13.1 | 2.2 | 18.8 | 1.9 |
| heparin | | | | | | |
| 2 | 142.7 | 74.3 | 777.9 | 152.0 | 266.3 | 35.5 |
| 2 + | 55.8 | 23.9 | 24.5 | 8.8 | 26.0 | 6.1 |
| heparin | | | | | | |
| Control | 7.6 | 0.4 | 8.4 | 0.2 | 8.2 | 0.1 |

| | |
|---|---|
| Human XCL2 | Mouse XCL1 |

| Antibody | MFI | +/− | MFI | +/− |
|---|---|---|---|---|
| 1 | 148.9 | 70.6 | 49.0 | 19.3 |
| 1 + | 21.8 | 6.3 | 9.2 | 0.1 |
| heparin | | | | |
| 2 | 767.7 | 289.6 | 61.3 | 12.5 |
| 2 + | 57.3 | 26.9 | 12.8 | 0.3 |
| heparin | | | | |
| Control | 8.6 | 0 | 8.0 | 0.1 |

The results above demonstrate that Antibodies 1 and 2 bind to inactive or dimeric XCL1 at the cell surface, and taken together with the results of Example 6, demonstrate that Antibodies 1 and 2 may bind inactive XCL1, sequestering it so that it does not activate XCR1.

### Example 10: XCL1 Specificity of Exemplified Antibodies

A human serum binding assay was used to identify non-specific targets of Antibodies 1 and 2, and MBC. Each antibody was coated in a microwell followed by treatment with serum. After an overnight incubation, unbound serum proteins were washed away prior to elution of antibody-bound proteins. These samples were digested with trypsin with peptides identified by mass spectrometry.

Antibodies 1 and 2 did not bind to other chemokines, but MBC was found to bind a substantial amount of a second, off-target chemokine. Subsequent experiments demonstrated that MBC lost biological activity in the presence of high concentrations of this chemokine (that is, similar to that expected in the physiological context) whereas the other Antibodies 1 and 2 were unaffected.

1. An antibody that binds human XCL1 comprising a heavy chain variable region (HCVR) and a light chain variable region (LCVR), wherein the HCVR comprises complementarity determining regions (CDRs) HCDR1, HCDR2, and HCDR3 and the LCVR comprises CDRs LCDR1, LCDR2, and LCDR3, wherein,
the HCDR1 comprises SEQ ID NO: 10,
the HCDR2 comprises SEQ ID NO: 11,
the HCDR3 comprises SEQ ID NO: 12, wherein Z is selected from Q and E,
the LCDR1 comprises SEQ ID NO: 5, wherein X is selected from S and D,
the LCDR2 comprises SEQ ID NO: 8, and
the LCDR3 comprises SEQ ID NO: 9.

2. The antibody of claim 1, wherein HCDR1 comprises SEQ ID NO: 10; HCDR2 comprises SEQ ID NO: 11; HCDR3 comprises SEQ ID NO: 13; LCDR1 comprises SEQ ID NO: 6; LCDR2 comprises SEQ ID NO: 8; and LCDR3 comprises SEQ ID NO: 9.

3. The antibody of claim 1, wherein the LCVR comprises SEQ ID NO: 17 and the HCVR comprises SEQ ID NO: 15, wherein Xₐ of SEQ ID NO: 15 is selected from Q and pyroglutamic acid.

4. The antibody of claim 3, wherein Xₐ is Q.

5. The antibody of claim 1, wherein the antibody comprises a heavy chain (HC) and a light chain (LC), wherein the HC comprises SEQ ID NO: 19, wherein X₁ of SEQ ID NO: 19 is selected from Q and pyroglutamic acid; X₂ of SEQ ID NO: 19 is G or absent; and X₃ of SEQ ID NO: 19 is K or absent; and the LC comprises SEQ ID NO: 21.

6. The antibody of claim 5, wherein X₁ is Q; X₂ is G; and X₃ is K.

7. The antibody of claim 1, wherein HCDR1 comprises SEQ ID NO: 10; HCDR2 comprises SEQ ID NO: 11; HCDR3 comprises SEQ ID NO: 14; LCDR1 comprises SEQ ID NO: 7; LCDR2 comprises SEQ ID NO: 8; and LCDR3 comprises SEQ ID NO: 9.

8. The antibody of claim 1, wherein the LCVR comprises SEQ ID NO: 18 and the HCVR comprises SEQ ID NO: 16, wherein Xₐ of SEQ ID NO: 16 is selected from Q and pyroglutamic acid.

9. The antibody of claim 8, wherein Xₐ is Q.

10. The antibody of claim 1, wherein the antibody comprises a heavy chain (HC) and a light chain (LC), wherein the HC comprises SEQ ID NO: 20, wherein X₁ of SEQ ID NO: 20 is selected from Q and pyroglutamic acid; X₂ of SEQ ID NO: 20 is G or absent; and X₃ of SEQ ID NO: 20 is K or absent; and the LC comprises SEQ ID NO: 22.

11. The antibody of claim 10, wherein X₁ is Q; X₂ is G; and X₃ is K.

12. The antibody of claim 1, wherein the antibody binds human XCL2.

13. The antibody of claim 1, wherein the antibody binds dimeric XCL1.

14. The antibody of claim 1, wherein the antibody binds XCL1 in a β-sheet configuration.

15. The antibody of claim 1, wherein XCL1 binds to glycosaminoglycan on a surface of a cell.

16. The antibody of claim 1, wherein the antibody binds dimeric XCL2.

17. The antibody of claim 16, wherein the antibody binds at least one amino acid residue on each monomeric unit of dimeric XCL2.

18. The antibody of claim 16, wherein XCL2 binds to glycosaminoglycan on a surface of a cell.

19. An antibody that binds human XCL1, comprising a heavy chain (HC) having at least 95% sequence identity to SEQ ID NO: 19 and SEQ ID NO: 20, and a light chain (LC) having at least 95% sequence identity to SEQ ID NO: 21 and SEQ ID NO: 22.

20. The antibody of any one of claim 1, wherein the antibody has a human IgG1 or IgG4 isotype.

21. A pharmaceutical composition comprising an antibody of claim 1 and one or more pharmaceutically acceptable carriers, diluents or excipients.

22. A method of treating alopecia areata, vitiligo, asthma, psoriasis, multiple sclerosis, psoriasis, rheumatoid arthritis, or ulcerative colitis in a subject in need thereof, comprising administering to the subject an effective amount of an antibody of claim 1 or a pharmaceutical composition of claim 21.

23. A nucleic acid comprising a sequence encoding SEQ ID NO: 23, SEQ ID NO: 24, SEQ ID NO: 25, or SEQ ID NO: 26.

24. A vector comprising a first nucleic acid sequence encoding SEQ ID NO: 23 or SEQ ID NO: 25 and a second nucleic acid sequence encoding SEQ ID NO: 24 or SEQ ID NO: 26.

25. A cell comprising the vector of claim 24.

26. A method of producing an antibody comprising culturing the cell of claim 25 under conditions such that the antibody is expressed and recovering the expressed antibody from the culture medium.


SEQUENCE LISTING (33 sequences)

SEQ ID NO: 1 - protein, 93 aa, Homo sp.
VGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 2 - protein, 93 aa, Homo sapiens
VGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 3 - protein, 93 aa, synthetic construct
VGSEVSDKRTCVSLTTQRLPCSRIKTYTITEGSLRAVIFITKRGLKVCADPQATWVRDCVRSMDRKSNTRNNMIQTKPTGTQQSTNTAVTLTG

SEQ ID NO: 4 - protein, 93 aa, synthetic construct
VGSEVSDKRTCVSLTTQRLPVSRIKTYTITEGSLRCVIFITKRGLKVCCDPQATWVRDVVRSMDRKSNTRNNMIQTKPTGTQQSTNTAVTLTG

SEQ ID NO: 5 - protein, 11 aa, synthetic construct
  VARIANT 5: note = X is S or D
QASQXISDYLN

SEQ ID NO: 6 - protein, 11 aa, synthetic construct
QASQDISDYLN

SEQ ID NO: 7 - protein, 11 aa, synthetic construct
QASQSISDYLN

SEQ ID NO: 8 - protein, 8 aa, synthetic construct
YDASAKQT

SEQ ID NO: 9 - protein, 10 aa, synthetic construct
QHYDNLGYYT

SEQ ID NO: 10 - protein, 13 aa, synthetic construct
KASGYTFTSYYMV

SEQ ID NO: 11 - protein, 17 aa, synthetic construct
VIDPSKGSTNYAQKFQG

SEQ ID NO: 12 - protein, 12 aa, synthetic construct
  VARIANT 6: note = Z is Q or E
AREGLZLLAFDI

SEQ ID NO: 13 - protein, 12 aa, synthetic construct
AREGLELLAFDI

SEQ ID NO: 14 - protein, 12 aa, synthetic construct
AREGLQLLAFDI

SEQ ID NO: 15 - protein, 119 aa, synthetic construct
  VARIANT 1: note = X is Q or pyroglutamic acid
XVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLELLAFDIWGQGTMVTVSS

SEQ ID NO: 16 - protein, 119 aa, synthetic construct
  VARIANT 1: note = X is Q or pyroglutamic acid
XVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLQLLAFDIWGQGTMVTVSS

SEQ ID NO: 17 - protein, 108 aa, synthetic construct
DIQMTQSPSSLSASVGDRVTITCQASQDISDYLNWYQQKPGKAPKLLIYDASAKQTGVPSRFSGSGSGTDFTFTISSLQPEDIATYYCQHYDNLGYYTFGQGTKLEIK

SEQ ID NO: 18 - protein, 108 aa, synthetic construct
DIQMTQSPSSLSASVGDRVTITCQASQSISDYLNWYQQKPGKAPKLLIYDASAKQTGVPSRFSGSGSGTDFTFTISSLQPEDIATYYCQHYDNLGYYTFGQGTKLEIK

SEQ ID NO: 19 - protein, 449 aa, synthetic construct
  VARIANT 1: note = X1 is Q or pyroglutamic acid
  VARIANT 448: note = X2 is G or absent
  VARIANT 449: note = X3 is K or absent
XVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLELLAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 20 - protein, 449 aa, synthetic construct
  VARIANT 1: note = X1 is Q or pyroglutamic acid
  VARIANT 448: note = X2 is G or absent
  VARIANT 449: note = X3 is K or absent
XVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLQLLAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPXX

SEQ ID NO: 21 - protein, 215 aa, synthetic construct
DIQMTQSPSSLSASVGDRVTITCQASQDISDYLNWYQQKPGKAPKLLIYDASAKQTGVPSRFSGSGSGTDFTFTISSLQPEDIATYYCQHYDNLGYYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 215 aa, synthetic construct
DIQMTQSPSSLSASVGDRVTITCQASQSISDYLNWYQQKPGKAPKLLIYDASAKQTGVPSRFSGSGSGTDFTFTISSLQPEDIATYYCQHYDNLGYYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1347 bp, synthetic construct
  source 1..1347: mol_type = other DNA
caggtgcagctggtgcagtctggggctgaggtgaagaagcctggggcctcagtgaaggtttcctgcaaagcgtctggatacaccttcaccagctattatatggtctgggtgcgacaggcccctggacaagggcttgagtggatgggagtaatcgaccctagtaaaggtagcacaaactacgcacagaagttccagggcagagtcaccatgaccagggacacgtccacgagcacagtctacatggagctgagcagcctgagatctgaggacacggccgtgtattactgtgcgagagaaggactggaactacttgcttttgatatctggggccaagggacaatggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 24 - DNA, 645 bp, synthetic construct
  source 1..645: mol_type = other DNA
gacatccagatgacccagtctccatcctccctgtctgcatctgtaggagacagagtcaccatcacttgccaggcgagtcaggacataagcgactatttaaattggtatcagcagaaaccagggaaagcccctaagctcctgatctacgatgcttccgctaaacaaacaggggtcccatcaaggttcagtggaagtggatctgggacagattttactttcaccatcagcagcctgcagcctgaagatattgcaacatattactgtcaacattatgataatctcggatactacacttttggccaagggaccaagctggagatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 25 - DNA, 1347 bp, synthetic construct
  source 1..1347: mol_type = other DNA
caggtgcagctggtgcagtctggggctgaggtgaagaagcctggggcctcagtgaaggtttcctgcaaagcgtctggatacaccttcaccagctattatatggtctgggtgcgacaggcccctggacaagggcttgagtggatgggagtaatcgaccctagtaaaggtagcacaaactacgcacagaagttccagggcagagtcaccatgaccagggacacgtccacgagcacagtctacatggagctgagcagcctgagatctgaggacacggccgtgtattactgtgcgagagaaggactgcaactacttgcttttgatatctggggccaagggacaatggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 26 - DNA, 645 bp, synthetic construct
  source 1..645: mol_type = other DNA
gacatccagatgacccagtctccatcctccctgtctgcatctgtaggagacagagtcaccatcacttgccaggcgagtcagagcataagcgactatttaaattggtatcagcagaaaccagggaaagcccctaagctcctgatctacgatgcttccgctaaacaaacaggggtcccatcaaggttcagtggaagtggatctgggacagattttactttcaccatcagcagcctgcagcctgaagatattgcaacatattactgtcaacattatgataatctcggatactacacttttggccaagggaccaagctggagatcaaaagaactgtggcggcgccatctgtcttcatcttcccgccatctgatgagcagttgaaatccggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 27 - protein, 9 aa, synthetic construct
VSLTTQRLP

SEQ ID NO: 28 - protein, 12 aa, synthetic construct
RAVIFITKRGLK

SEQ ID NO: 29 - protein, 5 aa, synthetic construct
TQRLP

SEQ ID NO: 30 - protein, 119 aa, synthetic construct
QVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLELLAFDIWGQGTMVTVSS

SEQ ID NO: 31 - protein, 119 aa, synthetic construct
QVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLQLLAFDIWGQGTMVTVSS

SEQ ID NO: 32 - protein, 449 aa, synthetic construct
QVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLELLAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 449 aa, synthetic construct
QVQLVQSGAEVKKPGASVKVSCKASGYTFTSYYMVWVRQAPGQGLEWMGVIDPSKGSTNYAQKFQGRVTMTRDTSTSTVYMELSSLRSEDTAVYYCAREGLQLLAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK